(19) United States
(12) Patent Application Publication
(10) Pub. No.: US 2024/0165370 A1
(43) Pub. Date: May 23, 2024

(54) ENHANCING SLEEP QUALITY

(71) Applicant: Samsung Electronics Co., Ltd., Suwon-si (KR)

(72) Inventors: Vutha Va, Plano, TX (US); Hao Chen, Allen, TX (US); Doyoon Kim, Suwon (KR); Jianzhong Zhang, Dallas, TX (US); Joonhyun Lee, Seoul (KR)

(21) Appl. No.: 18/490,657

(22) Filed: Oct. 19, 2023

Related U.S. Application Data
(60) Provisional application No. 63/426,646, filed on Nov. 18, 2022.

(51) Int. Cl. A61M 21/00; A61B 5/00; G16H 10/60

(52) U.S. Cl. CPC A61M 21/00; A61B 5/4809; A61B 5/4812; A61B 5/4815; A61B 5/7275; G16H 10/60

(57) ABSTRACT

An apparatus for monitoring sleep quality includes a plurality of sensors, a plurality of actuator modules, a transceiver, and a processor operatively coupled with the plurality of sensor modules, the plurality of actuator modules, and the transceiver. The processor is configured to monitor a sleep session of a user utilizing the apparatus. To monitor the sleep session, the processor is further configured to monitor a sleep state of the user, monitor a sleep stage of the user, and monitor a sleep condition. The processor is further configured to select a sleep facilitating action, control the sleep facilitating action based on the monitoring, and collect data related to the sleep session, and update a sleep history database associated with the user based on the data related to the sleep session.

### CROSS-REFERENCE TO RELATED APPLICATION AND CLAIM OF PRIORITY

This application claims priority under 35 U.S.C. § 119(e) to U.S. Provisional Patent Application No. 63/423,646 filed on Nov. 18, 2022. The above-identified provisional patent application is hereby incorporated by reference in its entirety.

### TECHNICAL FIELD

This disclosure relates generally to consumer devices. More specifically, this disclosure relates to methods and apparatuses for enhancing sleep quality.

### BACKGROUND

Sleep has a critical impact on our health. Sufficiently long and good sleep quality is critical for waking up feeling refreshed and be energetic during the day. Yet, in modern societies across the globe, sleep deprivation is a common problem. For example, according to some sources, almost half of US adults report feeling sleepy between three to seven days per week. While the recommended sleep duration for adults (16-64) is 7-8 hours, approximately 35% of all US adults report average sleep duration of less than seven hours. This is a common trend observed all over the world. Apart from sleep duration, sleep quality is also very important. Sleep is a rather complicated biological process, and it is not static but rather a dynamic process. One typical night of sleep for an adult consists of four to six rounds of sleep cycle, which is composed of four sleep stages. The sleep cycles are not uniform, but on average a sleep cycle lasts about 90 minutes. For good quality of sleep, it is critical that the body progresses smoothly through those sleep cycles. Furthermore, certain events, such as sleep apnea, can also affect sleep quality.

### SUMMARY

This disclosure provides methods and apparatuses for enhancing sleep quality.

In one embodiment, an apparatus for monitoring sleep quality is provided. The apparatus includes a plurality of sensors, a plurality of actuator modules, a transceiver, and a processor operatively coupled with the plurality of sensor modules, the plurality of actuator modules, and the transceiver. The processor is configured to monitor a sleep session of a user utilizing the apparatus. To monitor the sleep session, the processor is further configured to monitor a sleep state of the user, monitor a sleep stage of the user, and monitor a sleep condition. The processor is further configured to select a sleep facilitating action, control the sleep facilitating action based on the monitoring, and collect data related to the sleep session. The data includes feedback collected from the user at an end of the sleep session, and data collected from the monitoring of the sleep session. The processor is further configured to update a sleep history database associated with the user based on the data related to the sleep session.

In another embodiment, a method of operating an apparatus for monitoring sleep quality is provided. The method includes monitoring a sleep session of a user. Monitoring of the sleep session includes monitoring a sleep state of the user, monitoring a sleep stage of the user, and monitoring a sleep condition. The method further includes selecting a sleep facilitating action, controlling the sleep facilitating action based on the monitoring, and collecting data related to the sleep session. The data includes feedback collected from the user at an end of the sleep session, and data collected from the monitoring of the sleep session. The method further includes updating a sleep history database associated with the user based on the data related to the sleep session.

In yet another embodiment, a non-transitory computer readable medium embodying a computer program is provided. The computer program includes program code that, when executed by a processor of a device, causes the device to monitor a sleep session of a user. To monitor the sleep session of the user, the computer program further includes computer readable program code that when executed causes at least one processing device to monitor a sleep state of the user, monitor a sleep stage of the user, and monitor a sleep condition. The computer program further includes program code that, when executed by a processor of a device, causes the device to select a sleep facilitating action, control the sleep facilitating action based on the monitoring, collect data related to the sleep session, and update a sleep history database associated with the user based on the data related to the sleep session. The data includes feedback collected from the user at an end of the sleep session, and data collected from the monitoring of the sleep session.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure and its advantages, reference is now made to the following description, taken in conjunction with the accompanying drawings, in which:

FIG. 1 illustrates an example of hardware components for a smart sleep chair divided into several main categories according to embodiments of the present disclosure;

FIG. 2 illustrates an example of software components for a smart sleep chair according to embodiments of the present disclosure;

FIG. 3A illustrates an example smart sleep chair according to embodiments of the present disclosure;

FIG. 3B illustrates an example electronic device according to embodiments of the present disclosure;

FIG. 4 illustrates a block diagram for an example smart sleep chair according to embodiments of the present disclosure;

FIG. 5 illustrates a block diagram for an example smart sleep chair according to embodiments of the present disclosure;

FIG. 6 illustrates a process for improving sleep quality according to embodiments of the present disclosure;

FIG. 7 illustrates a process for selecting and specifying actions to facilitate falling asleep according to embodiments of the present disclosure;

FIG. 8 illustrates a process for determining when to disable certain sleep aid actions according to embodiments of the present disclosure;

FIG. 9 illustrates a process for determining when to disable certain sleep aid actions according to embodiments of the present disclosure;

FIG. 10 illustrates a process for using wakeup time interval and sleep stage monitoring to determine when to disable sleep aid actions according to embodiments of the present disclosure;

FIG. 11 illustrates a process for wakeup posture and sleep stage monitoring to determine when to disable sleep aid actions according to embodiments of the present disclosure;

FIG. 12 illustrates a process for monitoring and managing a snoring event according to embodiments of the present disclosure;

FIG. 13 illustrates a process to optimize selection of reclining adjustment parameters for mitigating snoring according to embodiments of the present disclosure;

FIG. 14 illustrates a process for monitoring and managing sleep apnea according to embodiments of the present disclosure;

FIG. 15 illustrates a process for monitoring and managing sleep apnea according to embodiments of the present disclosure;

FIG. 16 illustrates a process providing a control mechanism for selecting actions to mitigate sleep apnea according to embodiments of the present disclosure;

FIG. 17 illustrates a process for monitoring and managing teeth grinding according to embodiments of the present disclosure;

FIG. 18 illustrates a process for a smart alarm leveraging passive sleep stage monitoring according to embodiments of the present disclosure;

FIG. 19 illustrates a process for a smart alarm with capability to actively induce the body to prepare for waking up according to embodiments of the present disclosure;

FIG. 20 illustrates an example process for identifying an incorrect sleep stage prediction to further train/fine tune a sleep stage classifier according to embodiments of the present disclosure;

FIG. 21 illustrates an example process for identifying an incorrect sleep stage prediction to further train/fine tune a sleep stage classifier according to embodiments of the present disclosure; and

FIG. 22 illustrates a method for enhancing sleep quality according to embodiments of the present disclosure.

### DETAILED DESCRIPTION

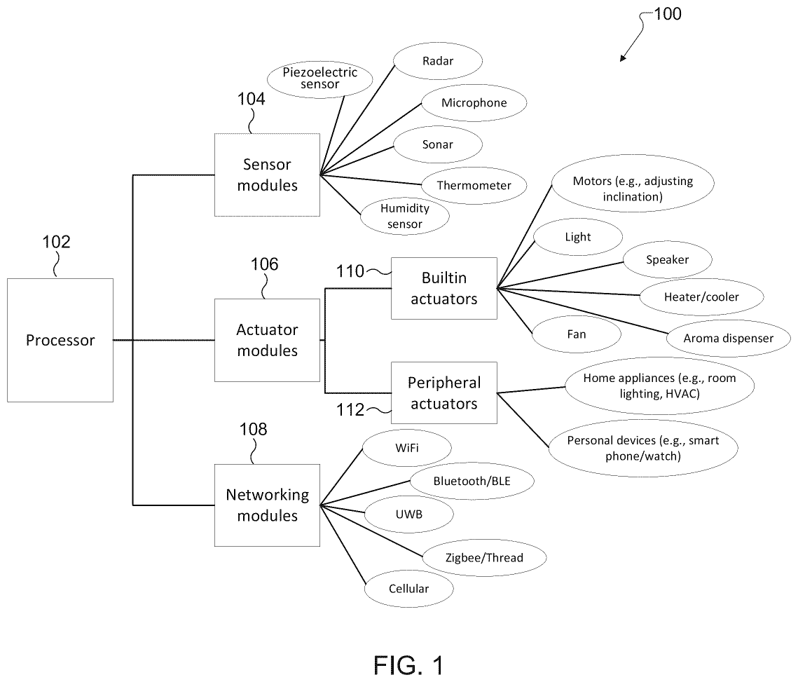

FIGS. 1 through 22, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged smart sleep chair.

In this disclosure, leveraging various non-invasive sensors (e.g., not restricting user's body movement and/or causing any discomfort), various methods and apparatuses are described to detect the sleep stage of a user as well as those events affecting the sleep quality. Additionally, per the detection and monitoring of the sleep stages and relevant events, methods and apparatuses to take certain actions to improve the user's sleep quality are disclosed. In this disclosure, a device that implements these capabilities may be referred to as a smart sleep chair. However, it should be understood that the disclosed methods and apparatuses may also be implemented in different forms such as a bed or a couch or similar furniture. Furthermore, it should be understood that the disclosed methods and apparatuses may also be implemented in different forms that allow the disclosed methods and apparatuses to be utilized as a peripheral device for standalone furniture, such as a chair, a bed, a couch, or similar.

A smart sleep chair as described herein may comprise two types of hardware components to implement the functionality of this disclosure.
1. Various non-invasive sensors for sleep stage and relevant events detection and monitoring: Some examples of these sensors include radar (at various radio frequencies), sonar sensors, micro-phone, temperature sensors, piezo-electric sensors, etc.
2. Various control devices that can take certain actions to improve sleep quality: Some examples include actuators that can adjust the inclination angle of the chair, speakers (e.g., playing some soothing music), electronics/appliance controllers (e.g., lighting, AC in the room etc.), wireless network capability that enables connection to peripherals (e.g., personal devices) to control and/or adjust some settings, etc.

FIG. 1 illustrates an example of hardware components 100 for a smart sleep chair divided into several main categories according to embodiments of the present disclosure. The embodiment of hardware components of FIG. 1 is for illustration only. Different embodiments of hardware component could be used without departing from the scope of this disclosure.

In the example of FIG. 1, the smart sleep chair has a main processor 102 that is connected to all other modules. It processes input from those modules, makes determinations of whether certain actions should be taken (e.g., aiming to improve sleep quality), and if actions are needed, it may output control signals to the appropriate entities. The components under the processor control may fall into one of three main categories:
1. Sensor modules 104: These are sensor devices that can be used to monitor the sleep stages and detect related events (e.g., limb/body movement, snoring, etc.). Examples of such sensors include radar, sonar, thermometer, humidity sensor, microphone, piezoelectric sensor, etc.
2. Actuator modules 106: These are devices that could influence the environment and can affect the user's sleep. One aim of a smart sleep chair is to determine proper actions allowable by these actuator devices to improve the user's sleep quality. Actuator modules may be further divided into two types: built-in and peripheral.
   a. Built-in actuators 110: These are devices equipped on the chair itself. Some examples include motors (e.g., for adjusting the reclining of the chair), speaker, lights, heater/cooler (e.g., something like an electric blanket or similar function could be embedded in the chair's cushion), fan, etc.
   b. Peripheral actuators 112: These are actuator devices in the vicinity of the chair (e.g., inside the room) that could be connected to the chair by the networking module. For example, these may include home appliances such as HVAC appliances, room lighting, air purifier, fan, etc. Another set of examples includes personal devices such as a smart phone or a smart watch (e.g., with the user's permission, the chair could change the setting to avoid disturbances from those devices).
3. Networking modules 108: These provide connectivity capability for the smart sleep chair. For example, the smart sleep chair may use networking modules to connect to the peripheral actuators. Examples of network modules include transceivers for WiFi, Bluetooth/BLE, Ultrawideband (UWB), Zigbee/Thread, cellular such as LTE/5G, etc. In some embodiments, networking modules may be used to connect the smart sleep chair to a remote apparatus, such as a cloud server on the internet, etc.

Although FIG. 1 illustrates one example of hardware components 100 for a smart sleep chair, various changes may be made to FIG. 1. For example, the smart sleep chair could include any number of each component shown in FIG. 1. Also, various components in FIG. 1 could be combined, further subdivided, or omitted and additional components could be added according to particular needs.

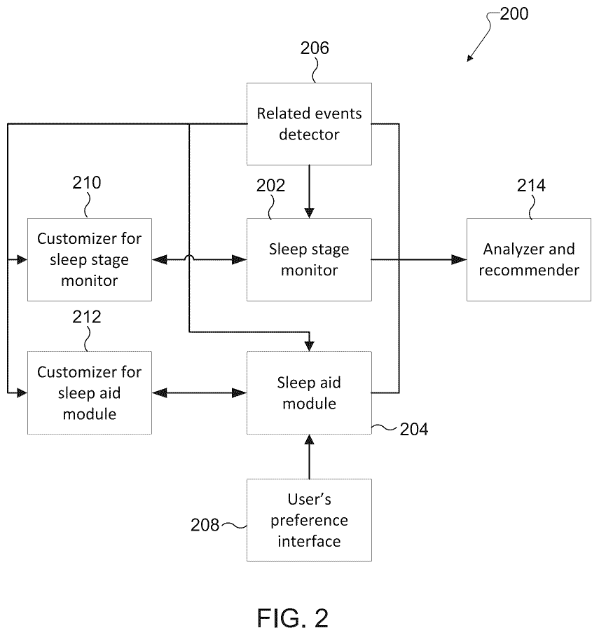

A primary purpose of a smart sleep chair as described herein is to monitor and improve the user's sleep quality. Software needed to support this goal may be divided into several components as illustrated in FIG. 2.

FIG. 2 illustrates an example 200 of software components for a smart sleep chair according to embodiments of the present disclosure. The embodiment of software components of FIG. 2 is for illustration only. Different embodiments of software components could be used without departing from the scope of this disclosure.

The core functionality of a smart sleep chair as described herein may lie in the sleep stage monitoring and the sleep aid module. The sleep aid module may allow the user to input their preference setting. The related events detector could be used to detect those events that may have impact on the sleep quality such as snoring, sleep apnea, etc. The customizers personalize the solutions (the sleep stage monitoring and sleep aid module) aiming at improving performance over time and adapt to the user's preferences and habits. Finally, the history from the sleep stage monitoring, the sleep aid module, and related events detector may be processed by the analyzer, which could provide some summary and related sleep quality metrics to the user. Further, those inputs may also be used to make recommendations to suggest some actions to the user that they may take to improve their sleep quality. Some details of the main functionalities of each component are provided below:
Sleep stage monitor (202): This module uses the sensing information from the sensor modules of the hardware component to detect and monitor the sleep state and sleep stage of the user. Sleep state may refer to, for example, the user being awake and attempting to sleep, actively sleeping, sleeping during a period where the user should be awakened, etc. Sleep stage may refer to the four sleep stages N1, N2, N3, and REM (Rapid Eye Movement). The sensing information may be processed into some intermediate format. For example, radar measurements may be processed to estimate the vital sign (e.g., breathing rate, heart rate, etc.) as well as other notable body movements.
Sleep aid module (204): This component uses the detected sleep states, sleep stages, related events, as well as user preference to take certain actions (e.g., by activating one or more of the actuators belonging to the actuator module) to facilitate and/or enhance the user's sleep quality. Note that the sleep aid module can operate at different states of the sleep including the duration for the user to fall asleep, when the user is asleep, as well as during the process of waking up.
Related events detector (206): This module is responsible for detecting various events that could affect the user's sleep quality. It uses the sensing information from the sensing module and may or may not use the same set of sensing information as used by the sleep stage monitoring module. Some examples of related events include snoring, limb/body movement, teeth grinding, night terror, sleep apnea, etc.
User's preference interface (208): As sleep habits vary a great deal for different individuals, different preferences for different users can be expected. For example, certain users may prefer to have light soothing music in the background to help them fall asleep, while other users may prefer complete silence. Similarly, some users may benefit from a preferred aromatic scent. These kinds of preferences that relate to ease of falling asleep and/or maintaining sleep quality could be provided through this interface.
Customizer for sleep stage monitor (210): Because of the large variation across users, it can be expected that there exists room for improvement for a generic solution (that aims at all or a large group of users). Such a generic solution is designed to work well for all targeted users, but may not be the best for any given user. Therefore, customization to tune and maximize the performance for the user could be beneficial. The purpose of this module is to collect related sensing information at times when it is determined that the detected sleep stage from the current solution might be incorrect. This is based on the fact that there are certain events that only occur in a specific sleep stage. Thus, if such an event is detected and the detected sleep stage is not consistent (i.e., that event could not occur in this sleep stage), then it is determined to be a likely wrong detection and this module would log the sensing information along with the expected sleep stage as the label. Such data could be used as additional training data personalized to the user, which could be used to fine-tune the classifier model to improve the performance.
Customizer for sleep aid module (212): The purpose of this module is similar to the customizer for sleep stage detector module, but with the focus on the sleep aid. Certain users may respond better to certain actions than others, and thus a one-size-fits-all solution may likely underperform. For example, in adjusting the chair reclining angle in response to a snoring event, the optimal angle would likely depend on the physique of the user as well as their personal preference. This module may request feedback from the user such as asking the user to rate the quality of the sleep session or some other related aspects. It may also use the sensing information (i.e., implicit feedback) to do the customization as well.
Analyzer and recommender (214): The detected sleep stages, related events, as well as responses from sleep aid module could be used to analyze the overall sleep quality. Such information may also be used to make certain recommendations for the user, for example, by providing some suggestions for adjusting their lifestyle that could help improve their sleep quality. Another aspect is that there are events that the smart sleep chair cannot respond to. E.g., if teeth grinding is detected, the recommender may suggest the user to consult a dentist and/or use a night guard.

Although FIG. 2 illustrates one example 200 of software components for a smart sleep chair, various changes may be made to FIG. 2. For example, the smart sleep chair could include any number of each component shown in FIG. 2. Also, various components in FIG. 2 could be combined, further subdivided, or omitted and additional components could be added according to particular needs.

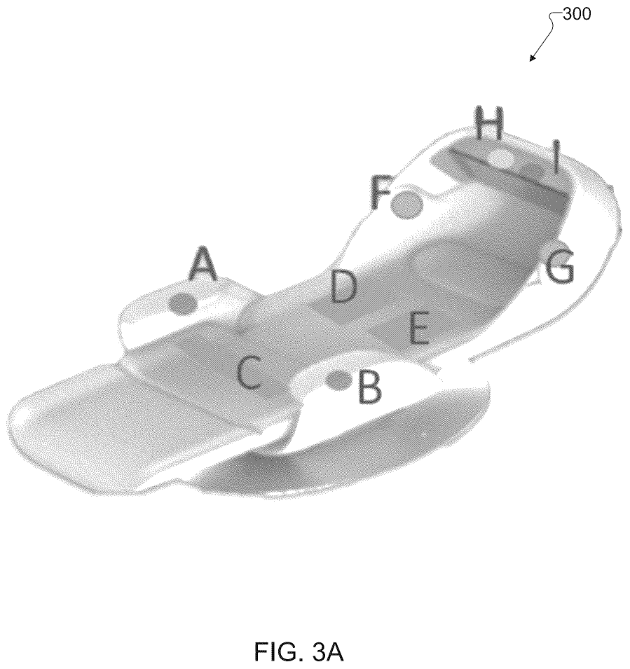

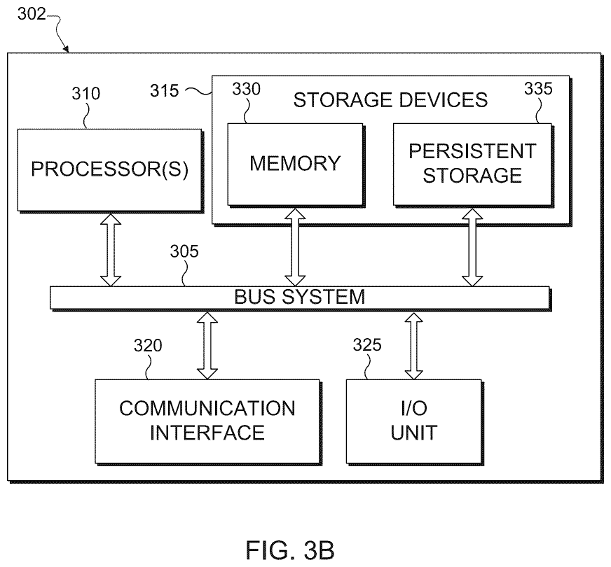

FIG. 3A illustrates an example smart sleep chair 300 according to embodiments of the present disclosure. The embodiment of a smart sleep chair 300 of FIG. 3A is for illustration only. Different embodiments of a smart sleep chair 300 could be used without departing from the scope of this disclosure.

The example of FIG. 3A, various sensors may be located in the potential locations of smart sleep chair 300 marked in FIG. 3A. In one embodiment, UWB sensors are located at locations A and I. In one embodiment, piezoelectric sensors located at locations D, E and C. In this embodiment, three piezoelectric sensors are deployed to further improve the performance. In one embodiment, mmWave (millimeter wave) sensors are located at locations F and G. In one embodiment, IR thermal sensors are located at location H. In one embodiment, Ultrasound radar is put on location F and G.

Although FIG. 3A illustrates one example of a smart sleep chair 300, various changes may be made to FIG. 3A. For example, the smart sleep chair 300 could include any number of each component described with respect to FIG. 3. Also, various components described with respect to FIG. 3A could be combined, further subdivided, located in alternative locations, or omitted and additional components could be added according to particular needs.

FIG. 3B illustrates an example electronic device according to embodiments of the present disclosure. In particular, FIG. 3B illustrates an example server 302 that may be operatively coupled to smart sleep chair 300 of FIG. 3A, and the server 302 could represent the processor 102 in FIG. 1. The server 302 can represent one or more processors, local servers, remote servers, clustered computers, and components that act as a single pool of seamless resources, a cloud-based server, and the like. The server 302 can be accessed by one or more of processor 102 and modules 104-108 of FIG. 3B or another server.

As shown in FIG. 3B, the server 302 includes a bus system 305 that supports communication between at least one processing device (such as a processor 310), at least one storage device 315, at least one communications interface 320, and at least one input/output (I/O) unit 325. The server 302 can represent one or more local servers, one or more remote servers, can be integrated directly into another apparatus such as smart sleep chair 300, or can be communicatively coupled with another apparatus such as smart sleep chair 300.

The processor 310 executes instructions that can be stored in a memory 330. The processor 310 can include any suitable number(s) and type(s) of processors or other devices in any suitable arrangement. Example types of processors 310 include microprocessors, microcontrollers, digital signal processors, field programmable gate arrays, application specific integrated circuits, and discrete circuitry.

The memory 330 and a persistent storage 335 are examples of storage devices 315 that represent any structure(s) capable of storing and facilitating retrieval of information (such as data, program code, or other suitable information on a temporary or permanent basis). The memory 330 can represent a random-access memory or any other suitable volatile or non-volatile storage device(s). For example, the instructions stored in the memory 330 can include instructions for enhancing sleep quality. The persistent storage 335 can contain one or more components or devices supporting longer-term storage of data, such as a read only memory, hard drive, flash memory, or optical disc.

The communications interface 320 supports communications with other systems or devices. For example, the communications interface 320 could include a network interface card or a wireless transceiver facilitating communications with networking modules 108 of FIG. 1. The communications interface 320 can support communications through any suitable physical or wireless communication link(s). For example, the communications interface 320 can transmit a bitstream containing user information another device such as smart sleep chair 300.

The I/O unit 325 allows for input and output of data. For example, the I/O unit 325 can provide a connection for user input through a keyboard, mouse, keypad, touchscreen, or other suitable input device. The I/O unit 325 can also send output to a display, printer, or other suitable output device. Note, however, that the I/O unit 325 can be omitted, such as when I/O interactions with the server 302 occur via a network connection.

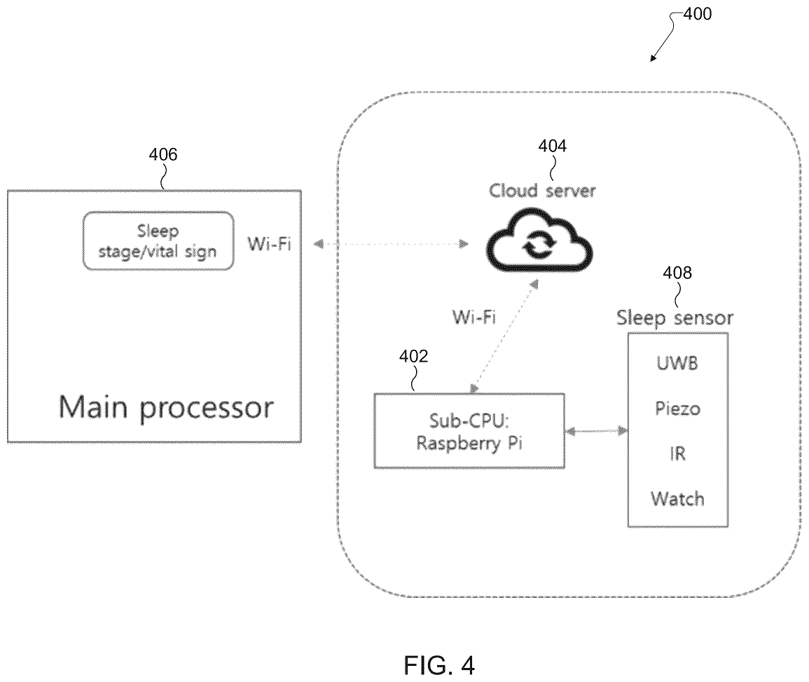

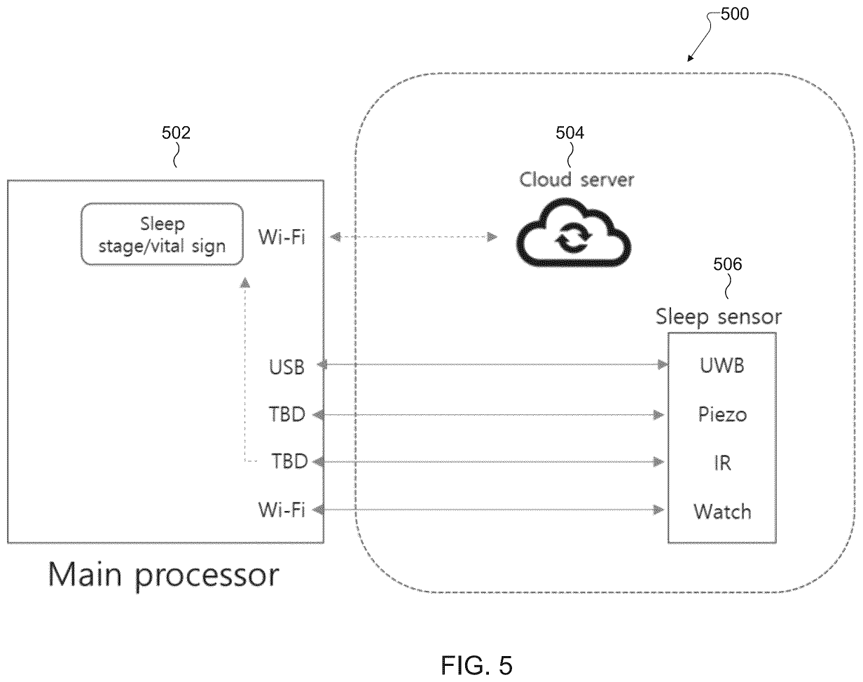

Note that while FIG. 3B may be described as representing the processor 102 of FIG. 1, the same or similar structure could be used in other devices or elements, including one or more of sub-CPU 402, cloud server 404, and main processor 406 of FIG. 4, and main processor 502 and cloud server 504 of FIG. 5. For example, cloud servers 404 and 504 could have the same or similar structure as that shown in FIG. 3B.

In one embodiment, as illustrated in FIG. 4, various sensors may interface to a sub-CPU.

FIG. 4 illustrates a block diagram for an example smart sleep chair 400 according to embodiments of the present disclosure. The embodiment of a smart sleep chair 400 of FIG. 4 is for illustration only. Different embodiments of a smart sleep chair 400 could be used without departing from the scope of this disclosure.

In the example of FIG. 4, the various sensors 408 interface with a sub-CPU 402 (e.g., a Raspberry Pi). The sub-CPU may be responsible for data and feature preprocessing. The sub-CPU may upload processed data and features over a networking module (e.g., Wi-Fi) to a cloud server 404. The cloud server may further process the features and generate sleep related detection results. The sleep related detection results may then be sent to a main processor 406 to drive related actions.

Although FIG. 4 illustrates a block diagram for one example of a smart sleep chair 400, various changes may be made to FIG. 4. For example, the smart sleep chair 400 could include any number of each component described with respect to FIG. 4. Also, various components described with respect to FIG. 4 could be combined, further subdivided, located in alternative locations, or omitted and additional components could be added according to particular needs.

In one embodiment, as illustrated in FIG. 5, various sensors may interface directly to a main processor.

FIG. 5 illustrates a block diagram for an example smart sleep chair 500 according to embodiments of the present disclosure. The embodiment of a smart sleep chair 500 of FIG. 5 is for illustration only. Different embodiments of a smart sleep chair 500 could be used without departing from the scope of this disclosure.

In the example of FIG. 5, the various sensors 506 interface with a main processor 502 directly. In one embodiment, the main processor may be responsible for data and feature preprocessing as well as generation of sleep stage information. In one embodiment, the main processor may upload processed data and features over a networking module (e.g., Wi-Fi) to a cloud server 504. The cloud server may further process the features and generate sleep related detection results such as sleep stage, sleep apnea detection, etc. The sleep related detection results may then be sent to a main processor to drive related actions.

Although FIG. 5 illustrates a block diagram for one example of a smart sleep chair 500, various changes may be made to FIG. 5. For example, the smart sleep chair 500 could include any number of each component described with respect to FIG. 5. Also, various components described with respect to FIG. 5 could be combined, further subdivided, located in alternative locations, or omitted and additional components could be added according to particular needs.

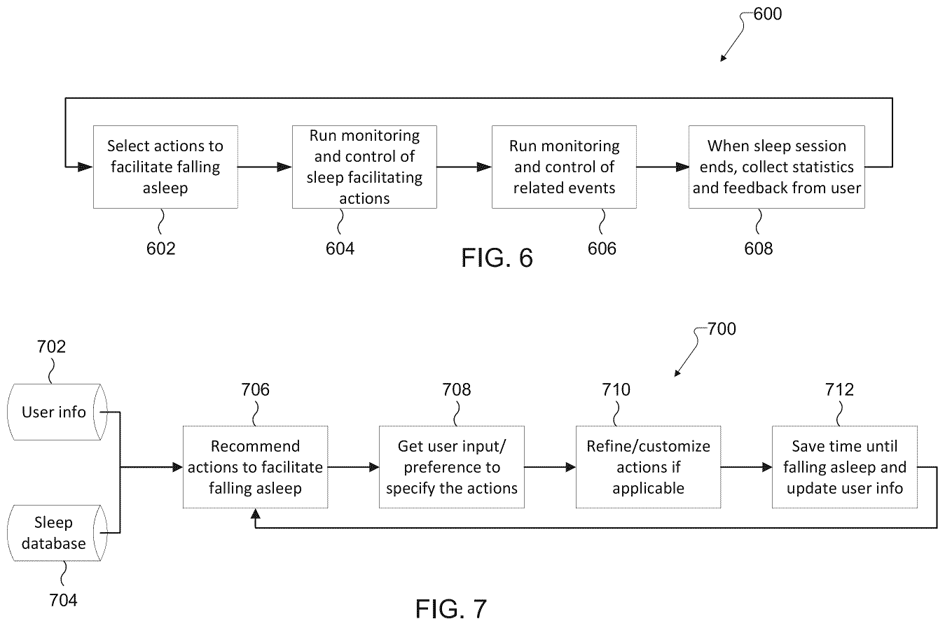

FIG. 6 illustrates a process 600 for improving sleep quality according to embodiments of the present disclosure. An embodiment of the process illustrated in FIG. 6 is for illustration only. One or more of the components illustrated in FIG. 6 may be implemented in specialized circuitry configured to perform the noted functions or one or more of the components may be implemented by one or more processors executing instructions to perform the noted functions. Other embodiments of a process 600 for improving sleep quality could be used without departing from the scope of this disclosure.

In the example of FIG. 6, an overall flow is illustrated for a solution for improving sleep quality related to a sleep aid module and a related customizer as described herein. Within the overall flow of the example of FIG. 6, there are four main components, where each component targets different aspects to improve the user's sleep quality. An overview of these four components are described below:
Solution to select actions to facilitate falling asleep (602): The purpose of this component is to select a set of actions that could be taken to adjust the environment such that it helps the user to fall asleep as quickly as possible. Some examples may include playing soothing music, adjusting the lighting, adjusting the room temperature (e.g., by adjusting a thermostat for an air conditioner), etc. The selection process may involve recommendation from the chair and the user's preferences.
Solution for monitoring and control of sleep facilitating actions (604): Depending on the sleep state, some changes to the actions to facilitate falling asleep may be helpful. For example, when the action to play music is selected, once the user is detected to be asleep (or after entering a particular sleep stage), the music may be turned off or the volume may be gradually reduced depending on the user.
Solution for monitoring and control of related events (606): Related events may refer to events that could have effects on sleep quality. Some examples include snoring, sleep apnea, teeth grinding, etc. When one or more of these events are detected, the chair may take a certain action or actions to mitigate or eliminate the problem, which may help improve sleep quality.
Statistics and user feedback collection (608): At the end of a sleep session, related statistics collected by the sensors and monitoring modules may be collected and may be further analyzed. Some examples of related information include the time taken to fall asleep, sleep duration overall (as well as breakdown into stages), etc. Also, some feedback from the user such as an overall rating (e.g., from 1-5 or 1-10 discrete values) as well as some certain aspects of the sleep session may be requested from the user and may be further analyzed. Note that to reduce the burden on the user, it is unnecessary to conduct a feedback request after every session, and requested feedback could be done on a certain schedule. The information collected here may be used to further refine/customize the above three modules with the aim of optimizing performance for the user.

Although FIG. 6 illustrates one example of a process 600 for improving sleep quality, various changes may be made to FIG. 6. For example, while shown as a series of steps, various steps in FIG. 6 could overlap, occur in parallel, occur in a different order, occur any number of times, or last for any duration.

FIG. 7 illustrates a process 700 for selecting and specifying actions to facilitate falling asleep according to embodiments of the present disclosure. An embodiment of the process illustrated in FIG. 7 is for illustration only. One or more of the components illustrated in FIG. 7 may be implemented in specialized circuitry configured to perform the noted functions or one or more of the components may be implemented by one or more processors executing instructions to perform the noted functions. Other embodiments of a process 700 for selecting and specifying actions to facilitate falling asleep could be used without departing from the scope of this disclosure.

FIG. 7 illustrates an overall flow of a procedure for the selection and refinement for specifying actions to facilitate falling asleep. In the example of FIG. 7, first, at steps 702 and 704, user information and a sleep history database are used to derive some recommendations on actions that could be helpful to the user for quickly falling asleep. The user information may include profile information such as age, gender, height, weight, body mass index (BMI), etc., that could provide information on the user physical conditions, and other sleep related preferences. The sleep history database as used here may have the user information from other users and a list of actions and their associated scores, where the score metric could be the time to fall asleep. Using the user information and the sleep history database, a machine learning solution or some statistical solution may be used to produce a ranking of the actions and those highly ranked actions could be recommended to the user at step 706. This list of highly-ranked actions may then be shown to the user to further make a selection and/or specification to reflect their preferences at step 708. For example, the user may accept or reject any given action in the list. For some actions, some further specification from the user is required at step 710. For example, consider the action for playing music or some soothing sound. The user's input to select a choice of music or sound along with a preferred volume could be obtained from the user. After this stage, there could be additional actions that could further be adjusted. For example, for an action to adjust the room temperature, the user may only provide a range, and in that case an RL (reinforcement learning)-based solution may be devised using a function of the time-till-asleep as the reward/cost signal, which would allow the automatic optimization (meaning no explicit intervention from the user) of the room temperature to minimize the time-till-asleep. Finally, relevant sensing information (e.g., the time-till-asleep) from the sleep session is logged, and this logged data could be attached or summarized to update the user information at step 712.

Some examples of actions that could help in facilitating the user to fall asleep more quickly include:
Playing music or some sound: The sound could be some soothing sound for the user such as nature sounds like raining sounds, some forest sounds, or even just white noise, or some audio recording such as an audio book.
Fan: For some users, a light breeze may induce sleep. Thus, activating a fan could be included as a possible action for facilitating falling asleep.
Adjusting lighting: Some users could prefer complete darkness, while other users might prefer dim lighting for optimal sleep conditions.
Adjusting room temperature: Room temperature is often an environmental parameter affecting user sleep, and thus could be adjusted to be optimal for the user's body type.
Silencing personal devices: With the user's authorization, the smart sleep chair may set some notification settings of the user's personal devices such as their phone, watch, etc. To avoid interruption to the sleep, once the user is detected to be in the smart sleep chair or in a certain sleep stage, those interrupting notifications may be disabled. Some exceptions may also be implemented to allow some critical notifications such as an emergency. This may include trusted public emergency alert or phone numbers, and it may also allow the user to record a list of numbers to be allowed through regardless (i.e., number of close and trusted family members and friends).
Preferred aroma: Certain scents could be helpful for some users to feel comfortable and ease falling asleep. In embodiments where a smart sleep chair includes an aroma dispenser, then this action may also be supported.
Preferred humidifier setting: This is another environmental parameter that can have impact on the user's sleep quality and could depend on the user body type and sleep habits. For example, if the user tends to breathe more from the mouth during sleep, then a higher relative humidity could be helpful for the user to avoid a dry mouth and throat.

Although FIG. 7 illustrates one example of a process 700 for selecting and specifying actions to facilitate falling asleep, various changes may be made to FIG. 7. For example, while shown as a series of steps, various steps in FIG. 7 could overlap, occur in parallel, occur in a different order, occur any number of times, or last for any duration.

Once the selection and specification of the actions to aid falling asleep are complete, the next stage is to perform monitoring and control of those selected actions in the sleep session. Note that this monitoring and control may not be applicable to all actions. For some actions, there may be no need for any changes during the sleep session. For example, if the lighting-related action was selected to be turn off all the lights, then there may be no reason to turn the lights back on in the middle of the sleep session. For some actions, some additional modification (e.g., like disabling those actions) could be performed based on the sleep stage monitoring.

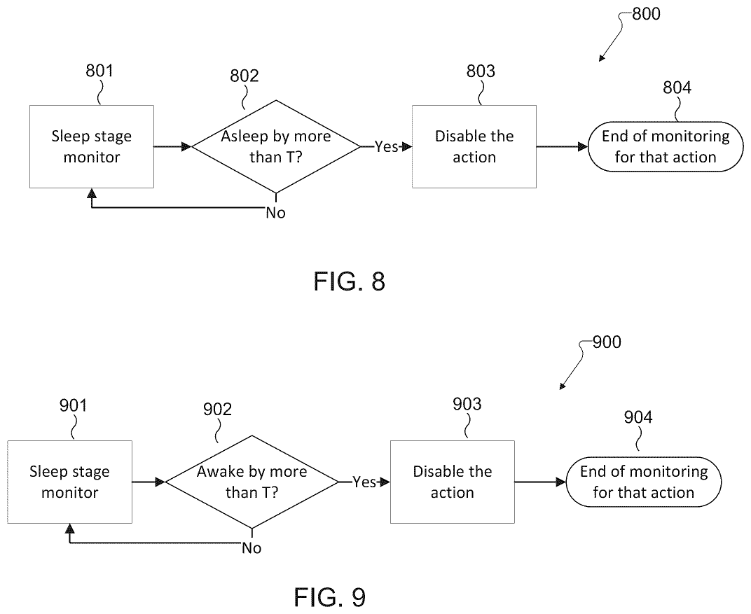

In one embodiment, similar as illustrated in FIG. 8, certain sleep aid actions may be disabled based on sleep stage monitoring results. For example, while certain actions may aid the user to fall asleep, the actions may interfere with sleep if the actions are active for too long. Some examples of these kinds of actions include music/soothing sound, lighting, aroma dispensing, etc.

FIG. 8 illustrates a process 800 for determining when to disable certain sleep aid actions according to embodiments of the present disclosure. An embodiment of the process illustrated in FIG. 8 is for illustration only. One or more of the components illustrated in FIG. 8 may be implemented in specialized circuitry configured to perform the noted functions or one or more of the components may be implemented by one or more processors executing instructions to perform the noted functions. Other embodiments of a process 800 for determining when to disable certain sleep aid actions could be used without departing from the scope of this disclosure.

In the example of FIG. 8, once the sleep session starts, a monitoring and control process may be defined for each action (those that needs to be disabled) separately. First, at step 801 the sleep stage is monitored. Based on the output of the sleep stage detector, it is determined at step 802 if the user has fallen asleep by at least a certain duration T (e.g., a threshold time). If the user is asleep by more than T, then the sleep aid action may be disabled at step 803. After disabling the action, at step 804 the monitoring for that action is complete and can be terminated. Note that variations of this embodiment could be conducted for better customization. For example, the asleep threshold duration T may be selected separately for different sleep facilitating actions. Also, the process of FIG. 8 is described as disabling, this needs not be a step transition. A smoother transition may be implemented instead for better user experience. For example, starting from when the user is detected to have fallen asleep by T sec, a volume of the music can start to decrease gradually until the music eventually becomes silent at time T+T₁, where T₁ is the duration for the gradual transition from enabled-state to disabled-state for the action (in this example music).

Although FIG. 8 illustrates one example of a process 800 for determining when to disable certain sleep aid actions, various changes may be made to FIG. 8. For example, while shown as a series of steps, various steps in FIG. 8 could overlap, occur in parallel, occur in a different order, occur any number of times, or last for any duration.

In one embodiment, similar as illustrated in FIG. 9, certain sleep aid actions may be disabled based on a sleep state of the user changing, such as the user waking up. One such action is the silencing of personal devices, which could help avoid sleep interruption, but could negatively affect the user's communication experience if they do not get notifications/alarms etc. when they are awake. Once the user has woken up, the notification setting can be returned to the normal mode (i.e., the user's own setting).

FIG. 9 illustrates a process 900 for determining when to disable certain sleep aid actions according to embodiments of the present disclosure. An embodiment of the process illustrated in FIG. 9 is for illustration only. One or more of the components illustrated in FIG. 9 may be implemented in specialized circuitry configured to perform the noted functions or one or more of the components may be implemented by one or more processors executing instructions to perform the noted functions. Other embodiments of a process 900 for determining when to disable certain sleep aid actions could be used without departing from the scope of this disclosure.

In the example of FIG. 9, once the sleep session starts, a monitoring and control process may be defined for each action (those that needs to be disabled) separately. First, at step 901 the sleep stage is monitored. Based on the output of the sleep stage detector, it is determined at step 902 if the user has been awake by at least a certain duration T (e.g., a threshold time). If the user is awake by more than T, then the sleep aid action may be disabled at step 903. After disabling the action, at step 904 the monitoring for that action is complete and can be terminated. Note that variations of this embodiment could be conducted for better customization. For example, the asleep threshold duration T may be selected separately for different sleep facilitating actions. Also, the process of FIG. 9 is described as disabling, this needs not be a step transition. A smoother transition may be implemented instead for better user experience. For example, starting from when the user is detected to have been awake by T sec, a brightness of lights can increase gradually until the lights reach a previously set brightness level at time T+T₁, where T₁ is the duration for the gradual transition from enabled-state to disabled-state for the action (in this example fading the lights on disabling a turning off the lights action).

Although FIG. 9 illustrates one example of a process 900 for determining when to disable certain sleep aid actions, various changes may be made to FIG. 9. For example, while shown as a series of steps, various steps in FIG. 9 could overlap, occur in parallel, occur in a different order, occur any number of times, or last for any duration.

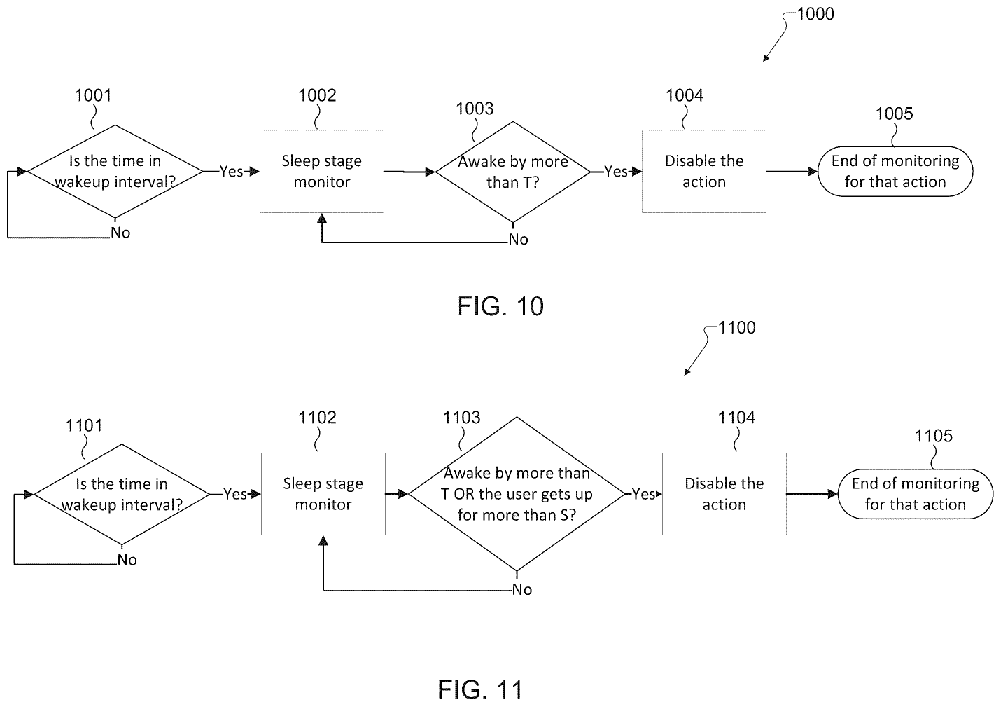

While the example embodiment of FIG. 9 relies solely on the sleep stage monitoring result, additional checks could be introduced for better user experience. For example, being awake in the middle of the night does not necessarily mean that the user is ready to wake up. The user might still want to get back to sleep. For such a situation, it would be better not to disable such action like silencing of personal devices, since alerts/notifications from the device(s) could make it harder for the user to fall back to sleep. It is desirable to try to properly gauge the intent of the user to wake up, and only then to disable the sleep aid action. Therefore, two additional checks may be used in combination with the sleep monitoring results, namely, the normal wake time and detection of whether the user gets up or off from the smart sleep chair. FIG. 10 shows an example where the wakeup interval is incorporated.

FIG. 10 illustrates a process 1000 for using wakeup time interval and sleep stage monitoring to determine when to disable sleep aid actions according to embodiments of the present disclosure. An embodiment of the process illustrated in FIG. 10 is for illustration only. One or more of the components illustrated in FIG. 10 may be implemented in specialized circuitry configured to perform the noted functions or one or more of the components may be implemented by one or more processors executing instructions to perform the noted functions. Other embodiments of a process 1000 for determining when to disable certain sleep aid actions could be used without departing from the scope of this disclosure.

In the example FIG. 10 a wakeup interval is incorporated to at step 1001 to determine when to disable sleep aid actions. The wakeup interval may refer to the time interval for typical waking up time for the user. For example, for a typical person, the usual waking up time could be between 6:00 to 9:00 in the morning. This time interval could be determined over time from a sleep routine of the user, or it could be set in the user's preference settings. At step 1002 the sleep stage is monitored. Based on the output of the sleep stage detector, it is determined at step 1003 if the user has been awake by at least a certain duration T (e.g., a threshold time). If the user is awake by more than T, then the sleep aid action may be disabled at step 1004. After disabling the action, at step 1005 the monitoring for that action is complete and can be terminated. Note that variations of this embodiment could be conducted for better customization. For example, the asleep threshold duration T may be selected separately for different sleep facilitating actions. Also, the process of FIG. 10 is described as disabling, this needs not be a step transition. A smoother transition may be implemented instead for better user experience. For example, starting from when the user is detected to have been awake by T sec, a brightness of lights can decrease gradually until the lights reach a previously set brightness level at time T+T₁, where T₁ is the duration for the gradual transition from enabled-state to disabled-state for the action (in this example fading the lights down disabling a turning on the lights action).

Although FIG. 10 illustrates one example of a process 1000 for using wakeup time interval and sleep stage monitoring to determine when to disable sleep aid actions, various changes may be made to FIG. 10. For example, while shown as a series of steps, various steps in FIG. 10 could overlap, occur in parallel, occur in a different order, occur any number of times, or last for any duration.

In another example embodiment as illustrated in FIG. 11, the detection of a wakeup posture may also be used to determine when to disable sleep aid actions.

FIG. 11 illustrates a process 1100 for wakeup posture and sleep stage monitoring to determine when to disable sleep aid actions according to embodiments of the present disclosure. An embodiment of the process illustrated in FIG. 11 is for illustration only. One or more of the components illustrated in FIG. 11 may be implemented in specialized circuitry configured to perform the noted functions or one or more of the components may be implemented by one or more processors executing instructions to perform the noted functions. Other embodiments of a process 1100 for determining when to disable certain sleep aid actions could be used without departing from the scope of this disclosure.

In the example of FIG. 11, wakeup posture may refer to situations such as when the user gets up and changes to a sitting position on the smart sleep chair or even getting off of the smart sleep chair altogether. To avoid the case where the user might be getting off briefly (e.g., like going to the rest room), here rather than an instantaneous get-up event, we consider the state of getting up by at least a duration S.

In the example FIG. 11 a wakeup interval is incorporated to at step 1101 to determine when to disable sleep aid actions. The wakeup interval may refer to the time interval for typical waking up time for the user. For example, for a typical person, the usual waking up time could be between 6:00 to 9:00 in the morning. This time interval could be determined over time from a sleep routine of the user, or it could be set in the user's preference settings. At step 1102 the sleep stage is monitored. Based on the output of the sleep stage detector, it is determined at step 1103 if the user has been awake by at least a certain duration T (e.g., a threshold time) or gets up for more than a certain duration S (e.g., a threshold time). If the user is awake by more than T or gets up for more than S, then the sleep aid action may be disabled at step 1104. After disabling the action, at step 1105 the monitoring for that action is complete and can be terminated. Note that variations of this embodiment could be conducted for better customization. For example, the asleep threshold duration T may be selected separately for different sleep facilitating actions. Also, the process of FIG. 10 is described as disabling, this needs not be a step transition. A smoother transition similarly as previously described may be implemented.

Although FIG. 11 illustrates one example of a process 1100 for using wakeup posture and sleep stage monitoring to determine when to disable sleep aid actions, various changes may be made to FIG. 11. For example, while shown as a series of steps, various steps in FIG. 11 could overlap, occur in parallel, occur in a different order, occur any number of times, or last for any duration.

In one embodiment, a smart sleep chair may monitor for an undesired sleep condition. For example, the smart sleep chair may monitor for an uncomfortable temperature in the room, whether the user is snoring, etc. and then perform a sleep aid action to improve the sleeping condition of the user.

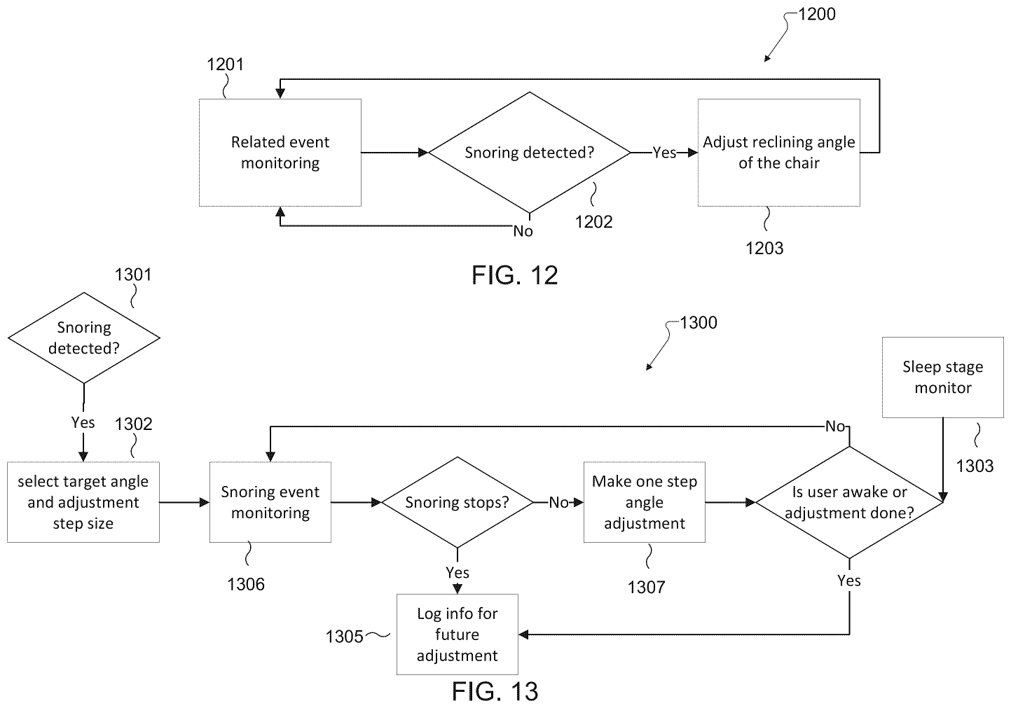

In one embodiment, similar as illustrated in FIG. 12, a snoring event is monitored and mitigated if snoring is detected.

FIG. 12 illustrates a process 1200 for monitoring and managing a snoring event according to embodiments of the present disclosure. An embodiment of the process illustrated in FIG. 12 is for illustration only. One or more of the components illustrated in FIG. 12 may be implemented in specialized circuitry configured to perform the noted functions or one or more of the components may be implemented by one or more processors executing instructions to perform the noted functions. Other embodiments of a process 1200 for monitoring and managing a snoring event could be used without departing from the scope of this disclosure.

In the example of FIG. 12, once the user falls asleep, a related event monitoring module can be activated at step 1201, and if a snoring event is detected at step 1202, some adjustment to the reclining angle of the chair may be conducted at step 1203 to help improve the airflow of the user, which could help mitigate the snoring. Since this adjustment to the angle is more personal due to the physiological differences for each user, the adjustment process could be learned, for example, using a reinforcement learning (RL) framework. The reclining angle adjustment may be decomposed into two parameters: the target reclining angle and the angle adjustment speed.

Although FIG. 12 illustrates one example of a process 1200 for monitoring and managing a snoring event, various changes may be made to FIG. 12. For example, while shown as a series of steps, various steps in FIG. 12 could overlap, occur in parallel, occur in a different order, occur any number of times, or last for any duration.

In one embodiment, the adjustment is done for a fixed angle adjustment step size, starting from the current angle. An example process is illustrated in FIG. 13.

FIG. 13 illustrates a process 1300 to optimize selection of reclining adjustment parameters for mitigating snoring according to embodiments of the present disclosure. An embodiment of the process illustrated in FIG. 13 is for illustration only. One or more of the components illustrated in FIG. 13 may be implemented in specialized circuitry configured to perform the noted functions or one or more of the components may be implemented by one or more processors executing instructions to perform the noted functions. Other embodiments of a process 1300 to optimize selection of reclining adjustment parameters for mitigating snoring could be used without departing from the scope of this disclosure.

In the example of FIG. 13, at the beginning when a snoring event is detected at step 1301 for the first time in a sleep session, at step 1302 the reclining adjustment solution first picks a target reclining angle and a reclining angle adjustment step size. As noted earlier, this reclining angle adjustment solution could be an RL-based solution. The reclining angle is then adjusted by the selected step size. While this adjustment is performed, at step 1303 the sleep state is monitored and checked whether the user might be woken up. If it is detected that the user is awake, at step 1305 the solution can log the information including the choices of the reclining adjustment parameters and the time where the awake state was detected. If the user is not detected to be awake, at step 1306 snoring event monitoring will continue until the next time step. If the snoring event is still detected, the angle adjustment step is continued at step 1307 until it reaches the target step. If snoring is no longer detected, then the relevant information is logged at step 1305. From an RL operation perspective, the reward function could be defined as follows:
If user becomes awake is detected, a reward of −1 is given.
If snoring is still detected after reaching the target reclining angle, a reward of 0 is given.
If snoring stops at any time before and including the target angle, a reward of 1 is given.

In another variation of the solution, the time until the snoring stops may also be used to define the reward. In this case, instead of always giving a reward of 1 when snoring stops at some time step before reaching the target angle, a monotonically increasing function of the inverse of the time till snoring stops may be used instead. That is the three reward cases above can be modified as follows (only the last one is modified):
If user becomes awake is detected, a reward of −1 is given.
If snoring is still detected after reaching the target reclining angle, a reward of 0 is given.
If snoring stops at before and including the target angle with a time until snoring stops t, a reward of ƒ(1/t) is given, where ƒ(⋅) is a monotonically increasing function.

Although FIG. 13 illustrates one example of a process 1300 to optimize selection of reclining adjustment parameters for mitigating snoring, various changes may be made to FIG. 13. For example, while shown as a series of steps, various steps in FIG. 13 could overlap, occur in parallel, occur in a different order, occur any number of times, or last for any duration.

Similar as illustrated in FIGS. 12-13, Another related event that could be monitored and managed is sleep apnea.

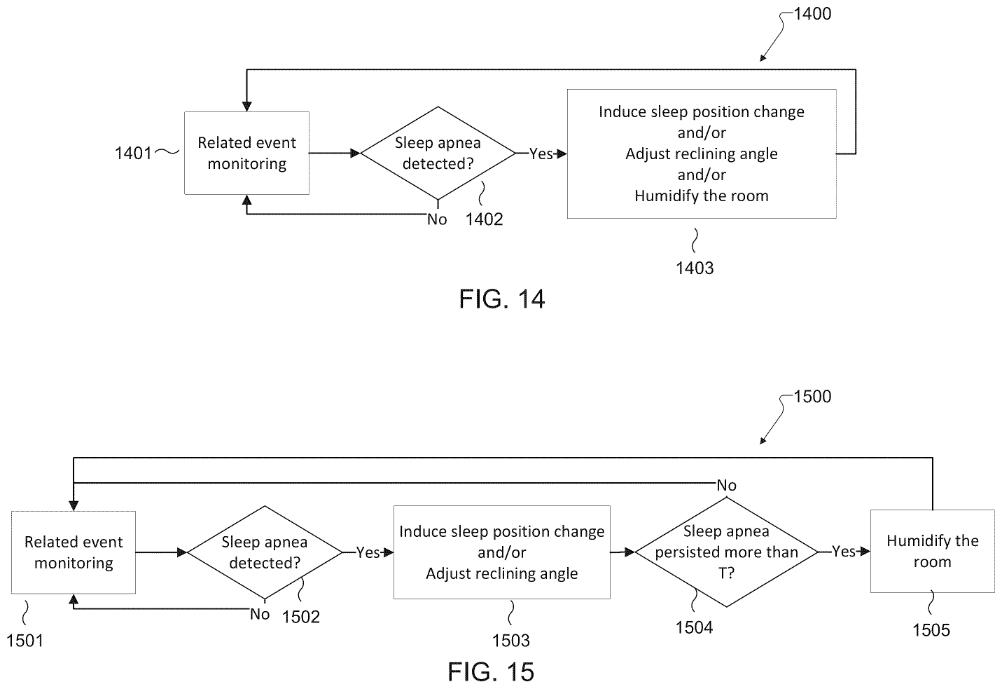

FIG. 14 illustrates a process 1400 for monitoring and managing sleep apnea according to embodiments of the present disclosure. An embodiment of the process illustrated in FIG. 14 is for illustration only. One or more of the components illustrated in FIG. 14 may be implemented in specialized circuitry configured to perform the noted functions or one or more of the components may be implemented by one or more processors executing instructions to perform the noted functions. Other embodiments of a process 1400 for monitoring and managing sleep apnea could be used without departing from the scope of this disclosure.

Sleep apnea is a common sleep disorder that causes a patient to stop breathing for short periods of time, which could negatively affect sleep quality. By monitoring the breathing cycle as well as oxygen levels (during the period of no breathing, the oxygen level drops), sleep apnea could be detected. Depending on the severity of sleep apnea, certain simple actions could help alleviate the symptoms. Some example actions include:
Changing sleep position
Adjusting the smart sleep chair reclining angle to align the user's spine
Humidifying the room to help reduce dry-mouth symptoms

In the example of FIG. 14, Once the user falls asleep, a related event monitoring module can be activated at step 1401, and if a sleep apnea event is detected at step 1402, some action (such as adjustment to the reclining angle of the chair) may be conducted at step 1403 to help mitigate the sleep apnea symptoms.

More details on how to apply the previously described actions are described with regard to FIG. 15. Changing sleep position and adjusting the reclining angle aim at reducing or eliminating the airway obstruction (thus sleep apnea), while increasing humidity is to reduce the consequences of sleep apnea (when sleep apnea persists).

Although FIG. 14 illustrates one example of a process 1400 for monitoring and managing sleep apnea, various changes may be made to FIG. 14. For example, while shown as a series of steps, various steps in FIG. 14 could overlap, occur in parallel, occur in a different order, occur any number of times, or last for any duration.

FIG. 15 illustrates a process 1500 for monitoring and managing sleep apnea according to embodiments of the present disclosure. An embodiment of the process illustrated in FIG. 15 is for illustration only. One or more of the components illustrated in FIG. 15 may be implemented in specialized circuitry configured to perform the noted functions or one or more of the components may be implemented by one or more processors executing instructions to perform the noted functions. Other embodiments of a process 1500 for monitoring and managing sleep apnea could be used without departing from the scope of this disclosure.

In the example of FIG. 15, Once the user falls asleep, a related event monitoring module can be activated at step 1501, and if a sleep apnea event is detected at step 1502, some action (such as adjustment to the reclining angle of the chair) may be conducted at step 1503 to help mitigate the sleep apnea symptoms. At step 1504, if the sleep apnea condition persists for at least a certain duration T, a humidifier is activated at step 1505.

Although FIG. 15 illustrates one example of a process 1500 for monitoring and managing sleep apnea, various changes may be made to FIG. 15. For example, while shown as a series of steps, various steps in FIG. 15 could overlap, occur in parallel, occur in a different order, occur any number of times, or last for any duration.

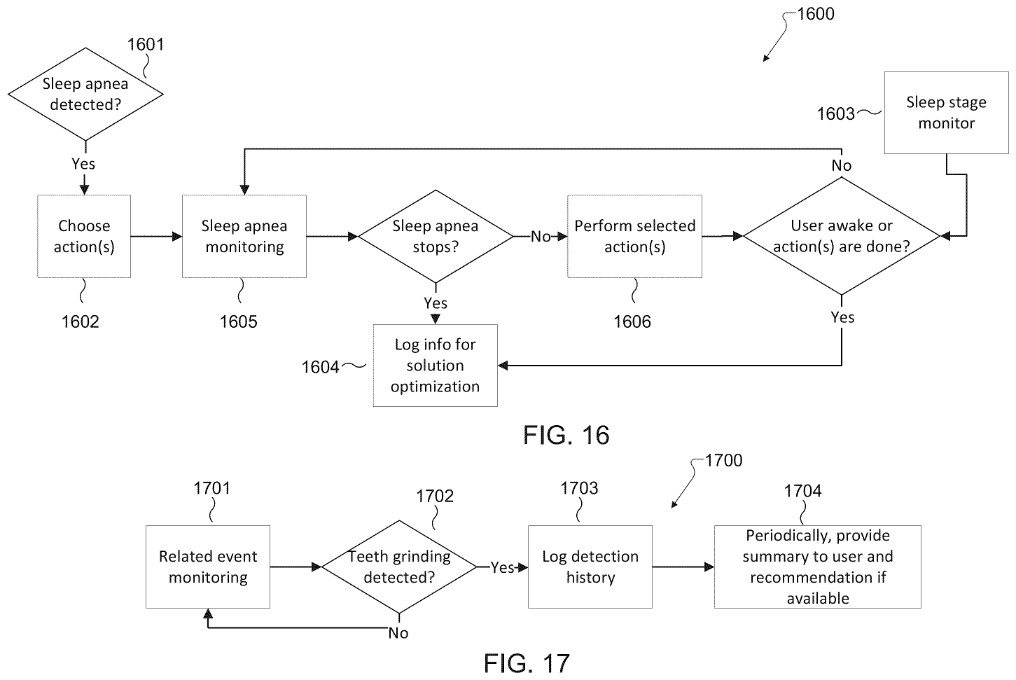

The determination of which combination of actions for mitigating sleep apnea (selecting one over another or selecting multiple) could be done similarly to the framework described for the embodiment of FIG. 13. An example is illustrated in FIG. 16.

FIG. 16 illustrates a process 1600 providing a control mechanism for selecting actions to mitigate sleep apnea according to embodiments of the present disclosure. An embodiment of the process illustrated in FIG. 16 is for illustration only. One or more of the components illustrated in FIG. 16 may be implemented in specialized circuitry configured to perform the noted functions or one or more of the components may be implemented by one or more processors executing instructions to perform the noted functions. Other embodiments of a process 1600 providing a control mechanism for selecting actions to mitigate sleep apnea could be used without departing from the scope of this disclosure.

In the example of FIG. 16, at step 1601, once sleep apnea is detected, the control to mitigate the condition is executed. First, at step 1602, the action is selected using for example a RL-based solution. After performing the selected action(s), at step 1603 it is checked if the user is woken due to the action(s) or that the selected action has been completed. If that is the case, at step 1604 the choice of action(s) and the fact that the user becomes awake is logged. If it is not the case (i.e., user still asleep and action not yet complete), at step 1605 then the process returns back to sleep apnea monitoring and continues the action at step 1606 for the next time step. If sleep apnea stops, then at step 1604 the related information is logged, and the control procedure is terminated. Performing the action selection may include a reward signal for the choice of the action. One choice of reward signal can be as follows:
If sleep apnea stops within some duration T, then a reward of 1 is given.
If sleep apnea persists more than the duration T, then a reward of −1 is given.

Note that for the action to adjust the reclining angle, a similar approach to the one used for snoring management could also be used. Particularly, different choices of the adjustment parameters (i.e., the target angle and reclining adjustment step size) could be treated as different actions. Also note that regarding the action to induce sleep position change, the mechanism could depend on the smart sleep chair capability. For example, some mechanical stimulus to one side of the cushion of the smart sleep chair (e.g., something similar to a massage chair) could be used. Another possibility is some temperature control of the smart sleep chair's cushion.

Regarding customization of the humidity setting, a possible solution is to use user feedback. One reason is that dry-mouth condition is difficult to detect using sensors. One example is to provide a pop-up rating question to the user when sleep apnea was detected. Also, the default setting could be set according to the user's preference and the setting may already be optimal or close to optimal.

Finally, the sleep apnea detection history could be logged and provided to the user as a health summary periodically (e.g., weekly). Another module could also be utilized that takes that history as an input to determine whether to recommend the user to seek further diagnosis and/or further treatment if it is determined to be severe.

Although FIG. 16 illustrates one example of a process 1600 providing a control mechanism for selecting actions to mitigate sleep apnea, various changes may be made to FIG. 16. For example, while shown as a series of steps, various steps in FIG. 16 could overlap, occur in parallel, occur in a different order, occur any number of times, or last for any duration.

Another related event that could be managed is teeth grinding, which could be detected using a microphone. For teeth grinding, there is no presently known real-time mitigating action that could be performed. In this case, the detection history may be logged and presented to the user in their health summary. In one embodiment, a health recommender solution may take the teeth grinding detection history as an input to determine the severity and whether to recommend some actions that could be taken by the user such as wearing a night guard or to consult a dentist to seek further treatment.

FIG. 17 illustrates a process 1700 for monitoring and managing teeth grinding according to embodiments of the present disclosure. An embodiment of the process illustrated in FIG. 17 is for illustration only. One or more of the components illustrated in FIG. 17 may be implemented in specialized circuitry configured to perform the noted functions or one or more of the components may be implemented by one or more processors executing instructions to perform the noted functions. Other embodiments of a process 1700 for monitoring and managing teeth grinding could be used without departing from the scope of this disclosure.

In the example of FIG. 17, first, at step 1701 an event related to teeth grinding is monitored. If it is determined at step 1702 that the user is grinding their teeth, at step 1703 the teeth grinding is log. At step 1704 a summary of the teeth grinding, and possible recommendations are provided to the user.

Although FIG. 17 illustrates one example of a process 1700 for monitoring and managing teeth grinding, various changes may be made to FIG. 17. For example, while shown as a series of steps, various steps in FIG. 17 could overlap, occur in parallel, occur in a different order, occur any number of times, or last for any duration.

In addition to being used to improve sleep quality, sleep stage monitoring capability provides new opportunities to improve the user's wake up experience as well. In one embodiment a smart alarm solution takes the current sleep stage of the user into account. It is known that depending on the sleep stage, the ease of being woken up as well as the wakeup experience can be quite different. For example, a person is more difficult to be woken up while in the N3 stage (i.e., deep sleep stage), and additionally, if woken up from the N3 stage, a person tends to feel disoriented and could have moderately impaired mental performance for 30 minutes to an hour. Therefore, it is best to wake the user up (e.g., by adjusting the timing to ring the alarm clock) at the end of the sleep cycle, when the sleep is lightest. This way, the user could feel more energetic and have a better wakeup experience.

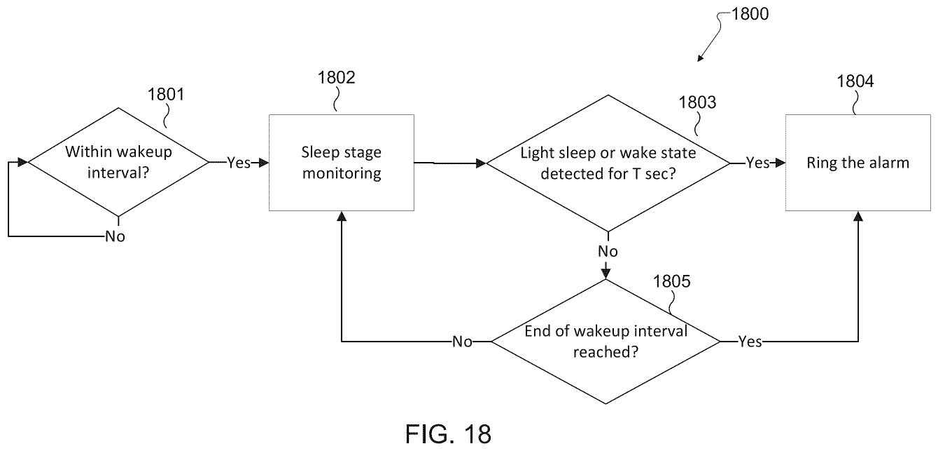

In one embodiment, similar as illustrated in FIG. 18, only passive monitoring is used to adjust the alarm clock timing. Since sleep cycle timing is not precise, in this case, the user is asked to set the target wakeup time as well as the allowable interval of time for waking up. For example, the user may set the target wakeup time as 7:00 AM with an allowable interval duration of 1 hour. In this case, the user allows the wake up time to be in the interval 6:30 AM-7:30 AM.

FIG. 18 illustrates a process 1800 for a smart alarm leveraging passive sleep stage monitoring according to embodiments of the present disclosure. An embodiment of the process illustrated in FIG. 18 is for illustration only. One or more of the components illustrated in FIG. 18 may be implemented in specialized circuitry configured to perform the noted functions or one or more of the components may be implemented by one or more processors executing instructions to perform the noted functions. Other embodiments of a process 1800 for a smart alarm leveraging passive sleep stage monitoring could be used without departing from the scope of this disclosure.

In the example of FIG. 18, first at step 1801, it is checked if the time is within the wakeup interval set by the user. If the time is within the wakeup interval set by the user, smart alarm timing control is executed leveraging a sleep stage monitoring capability. At step 1802, the sleep stage monitoring checks the sleep stage and determines at step 1803 whether the user has been awake or in light sleep stage for at least a duration T seconds. If this is true, it is determined that the user is in a proper state for waking up and the alarm can be activated at step 1804. If this is not the case, at step 1805 the time is checked against the end of the allowable interval. If the end of the interval is reached, then the alarm is activated regardless of the current sleep stage. While this may have some negative impact on the wakeup experience, the user schedule is also important.

Although FIG. 18 illustrates one example of a process 1800 for a smart alarm leveraging passive sleep stage monitoring, various changes may be made to FIG. 18. For example, while shown as a series of steps, various steps in FIG. 18 could overlap, occur in parallel, occur in a different order, occur any number of times, or last for any duration.

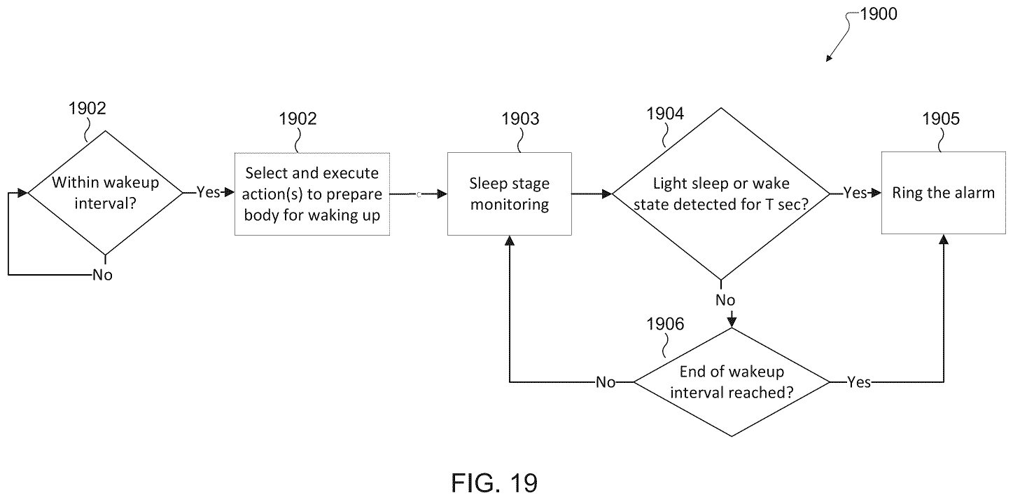

In another embodiment, similar as illustrated in FIG. 19, the smart sleep chair may have some capability to initialize some actions that help induce the body to prepare for waking up. Example actions include lighting, playing sounds (e.g., natural morning sounds such as birds chirping), adjusting the room temperature, etc.

FIG. 19 illustrates a process 1900 for a smart alarm with capability to actively induce the body to prepare for waking up according to embodiments of the present disclosure. An embodiment of the process illustrated in FIG. 19 is for illustration only. One or more of the components illustrated in FIG. 19 may be implemented in specialized circuitry configured to perform the noted functions or one or more of the components may be implemented by one or more processors executing instructions to perform the noted functions. Other embodiments of a process 1900 for a smart alarm with capability to actively induce the body to prepare for waking up could be used without departing from the scope of this disclosure.

In the example of FIG. 19, once it is determined that the present time is within the wake up interval, action(s) for inducing the body to wake up are executed and then a similar monitoring as in the example embodiment of Error! Reference source not found. is conducted. For the selection of those actions, a similar RL-based framework described in earlier embodiments could be used.

In the example of FIG. 19, first at step 1901, it is checked if the time is within the wakeup interval set by the user. If the time is within the wakeup interval set by the user, smart alarm timing control is executed leveraging a sleep stage monitoring capability. Once it is determined that the present time is within the wake up interval, at step 1902 action(s) for inducing the body to wake up are executed. For the selection of those actions, a similar RL-based framework described in earlier embodiments could be used. At step 1903, the sleep stage monitoring checks the sleep stage and determines at step 1904 whether the user has been awake or in light sleep stage for at least a duration T seconds. If this is true, it is determined that the user is in a proper state for waking up and the alarm can be activated at step 1905. If this is not the case, at step 1906 the time is checked against the end of the allowable interval. If the end of the interval is reached, then the alarm is activated regardless of the current sleep stage.

In one embodiment a reward function is defined. In one embodiment, a binary reward signal may be chosen as follows:
If the state ‘light sleep or awake state detected for T sec’ is met within the set wakeup interval, a reward signal of 1 is given.
If the state ‘light sleep or awake state detected for T sec’ is NOT met within the set wakeup interval, a reward signal of −1 is given.

With this binary reward signal, a multi-armed bandit solution could be applied. Note that since a combination of multiple actions could be allowed, a simple solution could be to treat the combination as an independent arm. A more advanced solution that tries to exploit the overlap could also be used.

In another embodiment, the reward signal could be selected to be the difference to the target wake up time (i.e., the center of the wakeup interval). That is, in this case, the time difference is treated as a kind of error or cost for learning algorithms to select the best action(s). Any variations of this error function such as some power of the difference may be used.

Although FIG. 19 illustrates one example of a process 1900 for a smart alarm with capability to actively induce the body to prepare for waking up, various changes may be made to FIG. 19. For example, while shown as a series of steps, various steps in FIG. 19 could overlap, occur in parallel, occur in a different order, occur any number of times, or last for any duration.

Each of the four sleep stages (N1, N2, N3, and REM) have their own characteristics and could have certain bodily events associated with each individual sleep stage. For example, a number of bodily events associated with specific sleep stages could be summarized as follows:
N2: N2 is when teeth grinding happens.
N3: Sleepwalking, night terrors, and bedwetting occur in N3.
REM: Dreaming and nightmares occur in REM. Also, during REM, the body is typically atonic, and becomes temporary paralyzed.

Some of these bodily events could be detectable by the sensors on the smart sleep chair, and could be used to improve the sleep stage detection solution. The detected events could be used in at least two scenarios to improve the accuracy of sleep stage detection:
1. The detected event could be used for gating the output of the original sleep stage monitoring solution (e.g., it could be a machine learning solution like a classifier). If the bodily event is a strong indicator for a certain sleep stage or it can rule out some stages to be unlikely, conflicting output from the sleep stage classifier could be overwritten.
2. For those gated cases, data could be collected, and those cases can be used to refine the classifier (e.g., by doing further of the classifier using the newly collected data once enough amount of data has been collected).

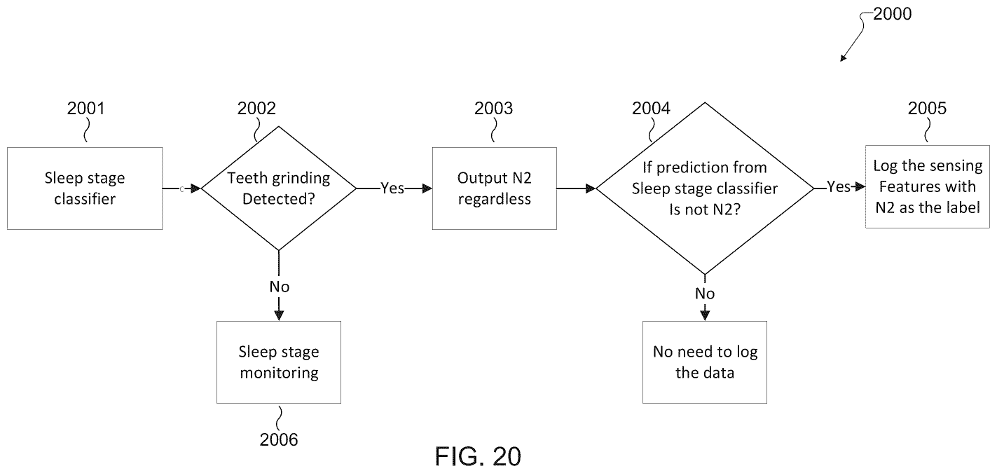

In one embodiment, similar as illustrated FIG. 20, a teeth grinding event is used as a strong indicator of the N2 sleep stage.

FIG. 20 illustrates an example process 2000 for identifying an incorrect sleep stage prediction to further train/fine tune a sleep stage classifier according to embodiments of the present disclosure. An embodiment of the process illustrated in FIG. 20 is for illustration only. One or more of the components illustrated in FIG. 20 may be implemented in specialized circuitry configured to perform the noted functions or one or more of the components may be implemented by one or more processors executing instructions to perform the noted functions. Other embodiments of a process 2000 for identifying an incorrect sleep stage prediction to further train/fine tune a sleep stage classifier could be used without departing from the scope of this disclosure.

In the example of FIG. 20, after doing prediction using the sleep stage classifier at step 2001, at step 2002 a teeth grinding event is checked. If teeth grinding is detected, at step 2003 N2 is output as the detected sleep stage regardless of the original prediction from the sleep stage classifier. Next, at step 2004 a check if the output of the sleep stage classifier is different from N2 is performed. If this is the case, this is an indication that the current sleep stage classifier's output is incorrect for this sample, and this sample could be used for future training/fine-tuning of the classifier. To do this, the input to the sleep stage classifier with N2 as the correct label is logged at step 2005. If teeth grinding is not detected, then the prediction from the sleep stage classifier is output as is at step 2006.

Although FIG. 20 illustrates one example of a process 2000 for identifying an incorrect sleep stage prediction to further train/fine tune a sleep stage classifier, various changes may be made to FIG. 20. For example, while shown as a series of steps, various steps in FIG. 20 could overlap, occur in parallel, occur in a different order, occur any number of times, or last for any duration.

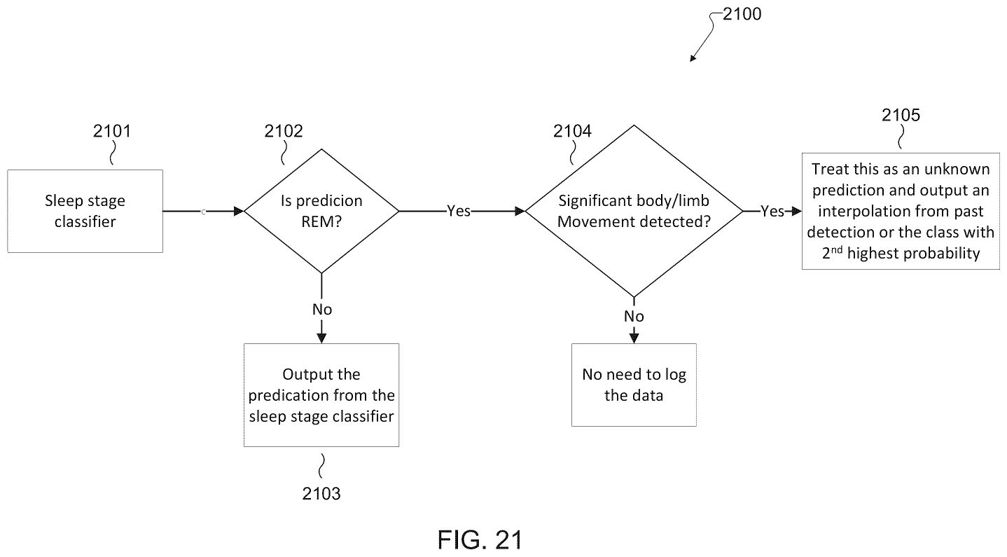

In one embodiment similar as illustrated FIG. 21, a body/limb movement event is used as a strong indicator against the REM sleep stage.

FIG. 21 illustrates an example process 2100 for identifying an incorrect sleep stage prediction to further train/fine tune a sleep stage classifier according to embodiments of the present disclosure. An embodiment of the process illustrated in FIG. 21 is for illustration only. One or more of the components illustrated in FIG. 21 may be implemented in specialized circuitry configured to perform the noted functions or one or more of the components may be implemented by one or more processors executing instructions to perform the noted functions. Other embodiments of a process 2100 for identifying an incorrect sleep stage prediction to further train/fine tune a sleep stage classifier could be used without departing from the scope of this disclosure.

The example of FIG. 21 relies on the fact that during REM our body typically becomes paralyzed. As such, if significant body/limb movement is detected, it is an indication that REM is unlikely. In this case, the fact could be used to negate some prediction. That is, if the sleep stage classifier predicts REM, but significant body/limb movement was detected, then it might be better to treat this as unknown class. For the purpose of outputting the predicted sleep stage, either interpolation from the past or use the 2nd highest probability of the prediction could be reasonable.

In the example of FIG. 21, after doing prediction using the sleep stage classifier at step 2101, at step 2102 it is checked if the sleep stage prediction is REM. If REM sleep is not detected, at step 2103 the predicted sleep stage from the sleep stage classifier is output. If REM sleep is detected, next, at step 2104 it is checked if significant body or limb movement is performed. If this is the case, this is an indication that the current sleep stage classifier is incorrect for this sample, and at step 2105 a better sleep stage prediction is output as previously discussed herein.

Although FIG. 21 illustrates one example of a process 2100 for identifying an incorrect sleep stage prediction to further train/fine tune a sleep stage classifier, various changes may be made to FIG. 21. For example, while shown as a series of steps, various steps in FIG. 21 could overlap, occur in parallel, occur in a different order, occur any number of times, or last for any duration.

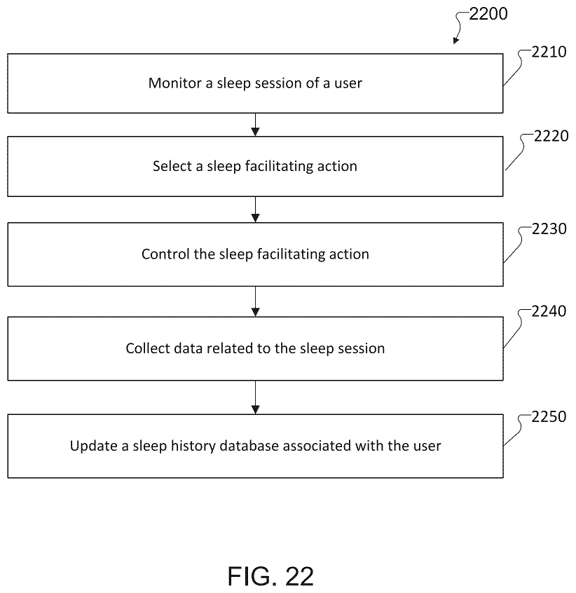

FIG. 22 illustrates a method 2200 for enhancing sleep quality according to embodiments of the present disclosure. An embodiment of the method illustrated in FIG. 22 is for illustration only. One or more of the components illustrated in FIG. 22 may be implemented in specialized circuitry configured to perform the noted functions or one or more of the components may be implemented by one or more processors executing instructions to perform the noted functions. Other embodiments of a method 2200 for enhancing sleep quality used without departing from the scope of this disclosure.

As illustrated in FIG. 22, the method 2200 begins at step 2210. At step 2210, a smart sleep chair monitors a sleep session of a user. The monitoring may include monitoring a sleep state of the user (e.g., whether the user is awake or asleep), monitoring a sleep stage of the user (e.g., N1, N2, N3 or REM), and monitoring a sleep condition (e.g., the ambient temperature, noise level, lighting level, etc.). At step 2220, the smart sleep chair selects a sleep facilitating action (e.g., dimming the lights, adjusting the position of the smart sleep chair, playing music, etc.). At step 2230, the smart sleep chair controls the sleep facilitating action based on the monitoring (e.g., turning off music after the user falls asleep). At step 2240, the smart sleep chair collects data related to the sleep session. The data includes feedback collected from the user at an end of the sleep session (e.g., collecting information regarding the user's preferences) and data collected from the monitoring of the sleep session (e.g., the timing of the user's sleep states and sleep stages). Finally, at step 2250, the smart sleep chair updates a sleep history database associated with the user based on the data related to the sleep session.

Although FIG. 22 illustrates one example of a method 2200 for enhancing sleep quality, various changes may be made to FIG. 22. For example, while shown as a series of steps, various steps in FIG. 22 could overlap, occur in parallel, occur in a different order, or occur any number of times.

Any of the above variation embodiments can be utilized independently or in combination with at least one other variation embodiment. The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claim scope. The scope of patented subject matter is defined by the claims.

What is claimed is:

1. An apparatus for monitoring sleep quality comprising:
a plurality of sensor modules;
a plurality of actuator modules;
a transceiver;
a processor operatively coupled with the plurality of sensor modules, the plurality of actuator modules, and the transceiver, the processor configured to:
monitor a sleep session of a user utilizing the apparatus, wherein to monitor the sleep session the processor is further configured to:
monitor a sleep state of the user,
monitor a sleep stage of the user, and
monitor a sleep condition;
select a sleep facilitating action;
control the sleep facilitating action based on the monitoring;
collect data related to the sleep session, wherein the data includes:
feedback collected from the user at an end of the sleep session, and
data collected from the monitoring of the sleep session; and
update a sleep history database associated with the user based on the data related to the sleep session.

2. The apparatus of claim 1, wherein to select the sleep facilitating action the processor is further configured to:
receive information related to a plurality of sleep facilitating actions;
receive information from the sleep history database; and
determine whether to modify at least one sleep facilitating action based on information received from the sleep history database.

3. The apparatus of claim 1, wherein to control the sleep facilitating action, the processor is further configured to:
determine whether the sleep state of the user has changed for longer than a threshold time; and
enable or disable the sleep facilitating action based on the sleep state of the user changing for longer than the threshold time.

4. The apparatus of claim 1, wherein to control the sleep facilitating action, the processor is further configured to:
detect an undesired sleep condition; and
enable or disable the sleep facilitating action based on detecting the undesired sleep condition.

5. The apparatus of claim 4, wherein the processor is further configured to:
determine whether the detected undesired sleep condition has persisted longer than a threshold time; and
enable or disable a second sleep facilitating action based on determining that the undesired sleep condition has persisted longer than the threshold time.

6. The apparatus of claim 1, wherein to control the sleep facilitating action, the processor is further configured to:
determine the sleep stage of the user; and
enable or disable the sleep facilitating action based on the sleep stage of the user.

7. The apparatus of claim 1, wherein the processor is further configured to:
determine that the sleep session has ended;
perform an analysis of the sleep session based on the collected data; and
provide feedback to the user based on the analysis.

8. The apparatus of claim 1, wherein the processor is further configured to:
predict a sleep stage of the user;
detect a sleep condition related to the user;
based on the detection of the sleep condition, determine whether the predicted sleep stage is accurate; and
collect data related to the detected sleep condition and the predicted sleep stage based on an accuracy of the predicted sleep stage.

9. A method of operating an apparatus for monitoring sleep quality, the method comprising:
monitoring a sleep session of a user, wherein monitoring of the sleep session further comprises:
monitoring a sleep state of the user;
monitoring a sleep stage of the user; and
monitoring a sleep condition;
selecting a sleep facilitating action;
controlling the sleep facilitating action based on the monitoring;
collecting data related to the sleep session, wherein the data includes:
feedback collected from the user at an end of the sleep session, and
data collected from the monitoring of the sleep session; and
updating a sleep history database associated with the user based on the data related to the sleep session.

10. The method of claim 9, wherein selecting the sleep facilitating action comprises:
receiving information related to a plurality of sleep facilitating actions;
receiving information from the sleep history database; and
determining whether to modify at least one sleep facilitating action based on information received from the sleep history database.

11. The method of claim 9, wherein controlling the sleep facilitating action comprises:
determining whether the sleep state of the user has changed for longer than a threshold time; and
enabling or disabling the sleep facilitating action based on the sleep state of the user changing for longer than the threshold time.

12. The method of claim 9, wherein controlling the sleep facilitating action comprises:
detecting an undesired sleep condition; and
enabling or disabling the sleep facilitating action based on detecting the undesired sleep condition.

13. The method of claim 12, further comprising:
determining whether the detected undesired sleep condition has persisted longer than a threshold time; and
enabling or disabling a second sleep facilitating action based on determining that the undesired sleep condition has persisted longer than the threshold time.

14. The method of claim 9, wherein controlling the sleep facilitating action comprises:
determining the sleep stage of the user; and
enabling or disabling the sleep facilitating action based on the sleep stage of the user.

15. The method of claim 9, further comprising:
determining that the sleep session has ended;
performing an analysis of the sleep session based on the collected data; and
providing feedback to the user based on the analysis.

16. The method of claim 9, further comprising:
predicting a sleep stage of the user;
detecting a sleep condition related to the user;
based on the detection of the sleep condition, determining whether the predicted sleep stage is accurate; and
collecting data related to the detected sleep condition and the predicted sleep stage based on an accuracy of the predicted sleep stage.

17. A non-transitory computer readable medium embodying a computer program, the computer program comprising program code that, when executed by a processor of a device, causes the device to:
monitor a sleep session of a user, wherein to monitor the sleep session the computer program further comprises computer readable program code that when executed causes at least one processing device to:
monitor a sleep state of the user;
monitor a sleep stage of the user; and
monitor a sleep condition;
select a sleep facilitating action;
control the sleep facilitating action based on the monitoring;
collect data related to the sleep session, wherein the data includes:
feedback collected from the user at an end of the sleep session, and
data collected from the monitoring of the sleep session; and
update a sleep history database associated with the user based on the data related to the sleep session.

18. The non-transitory computer readable medium of claim 17, wherein to select the sleep facilitating action, the computer program further comprises computer readable program code that when executed causes at least one processing device to:
receive information related to a plurality of sleep facilitating actions;
receive information from the sleep history database; and
determine whether to modify at least one sleep facilitating action based on information received from the sleep history database.

19. The non-transitory computer readable medium of claim 17, wherein the computer program further comprises computer readable program code that when executed causes at least one processing device to:
determine that the sleep session has ended;
perform an analysis of the sleep session based on the collected data; and
provide feedback to the user based on the analysis.

20. The non-transitory computer readable medium of claim 17, wherein the computer program further comprises computer readable program code that when executed causes at least one processing device to:
predict a sleep stage of the user;
detect a sleep condition related to the user;
based on the detection of the sleep condition, determining whether the predicted sleep stage is accurate; and
collect data related to the detected sleep condition and the predicted sleep stage based on an accuracy of the predicted sleep stage.
